(19) United States
(12) Patent Application Publication
(10) Pub. No.: US 2009/0298893 A1
(43) Pub. Date: Dec. 3, 2009

(54) Addition Salts of Tolperisone, Processes for Their Preparation and Use Thereof

(76) Inventors: Rudolf-Giesbert Alken, Sjoedala (SE); Frank Schneider, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 11/886,887

(22) PCT Filed: Mar. 21, 2006
(86) PCT No.: PCT/DE2006/000535

§ 371 Date: Aug. 12, 2009

(30) Foreign Application Priority Data
Mar. 25, 2005 (DE) 102005014080.7

(51) Int. Cl. A61K 31/44; C07D 211/82

(52) U.S. Cl. 514/358; 546/340

(57) ABSTRACT

Addition salts of 2,4′-dimethyl-3-piperidinopropiophenone (tolperisone) are described, wherein the salt is formed with an acid R—COOH and wherein R is the organic group of a physiologically compatible organic acid. Described also are processes for the preparation of these addition salts, the use thereof for pharmaceutical preparations and pharmaceuticals containing these addition salts.

The invention relates to addition salts of tolperisone, processes for their preparation, the use thereof for pharmaceutical preparations and pharmaceuticals containing these addition salts.

Tolperisone is the international free name for the muscle relaxant (RS)-2,4′-dimethyl-3-piperidinopropiophenone with the empirical formula C₁₆H₂₃NO.

Tolperisone and its salts find application in: painful spasms, tensing of the musculature, cervical syndrome, cervicobrachial syndrome, lumbar syndrome, osteoporosis, arthroses of the large joints, rheumatic diseases, fibromyalgia syndrome, chronic polyarthritis, overstressing caused by occupation and sports.

Tolperisone remains stable at low pH for a long time. In the basic pH region, however, color changes occur and the active ingredient is degraded. In order to assure the stability of tolperisone in pharmaceutical preparations, one utilizes addition salts of tolperisone with pharmaceutically acceptable acids.

JP 51128418 describes the preparation of the addition salt tolperisone hydrochloride.

WO 2004/050648 describes a process for the preparation of addition salts of tolperisone, in which 4-methylpropiophenone, piperidine hydrochloride and 1,2-dioxolane as well as an inorganic acid are utilized as the initial substances.

The preparation of optically active tolperisone is disclosed in JP 53040779. Different pharmacological effects of the enantiomers of tolperisone are described herein.

The tolperisone preparations found on the market at this time are stable for 3 years at most. If tolperisone hydrochloride is stored for a longer time, there can be a loss of hydrochloride due to evaporation. Because of this, the desired pH is not maintained and the stability of the tolperisone is no longer assured.

The object of the invention is to provide stable tolperisone addition salts, which possess a longer time of stability in comparison to tolperisone hydrochloride and can be used without hesitation in pharmaceutical preparations.

This object is accomplished with addition salts of tolperisone according to the principal claim. Advantageous embodiments of the addition salts according to the invention are characterized in the dependent claims.

One subject of the of the present invention thus involves addition salts of tolperisone (2,4′-dimethyl-3-piperidinopropiophenone) of Formula (A)

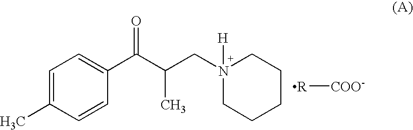

wherein

R is the organic group of a physiologically compatible organic acid.

According to the invention, it is preferred that R is an aliphatic, saturated or unsaturated group with up to 5 C atoms, which is optionally substituted with one or more hydroxy, oxo and/or carboxy groups.

In addition, it is preferred according to the invention that R is an aryl or aralkyl group, wherein the group contains 5 to 9 C atoms and is optionally substituted with one or more hydroxy and/or carboxy groups.

In addition, according to the invention, it is particularly preferred that the physiologically compatible organic acid is selected from acetic acid, propionic acid, malonic acid, oxalic acid, gluconic acid, succinic acid, maleic acid, fumaric acid, lactic acid, tartaric acid, malic acid, citric acid, pyruvic acid, hydroxybutyric acids, adipic acid, salicylic acid, phthalic acid, mandelic acid and benzoic acid.

According to the invention, it is most particularly preferred that the physiologically compatible organic acid is citric acid.

Particularly preferred are salts according to the invention of the general Formula (A), namely tolperisone citrate, (S)-tolperisone citrate or (R)-tolperisone citrate.

Most particularly preferred are salts in which the tolperisone is present in optically pure form as the (R)- or (S)-enantiomer.

Tolperisone has an asymmetry center in position 2, which leads to the corresponding (R)- or (S)-enantiomer. The subject of the present invention also includes all salts according to the invention containing organic acids in which the asymmetry center of the tolperisone is present in optically pure or optically enriched form, as well as all mixtures thereof, including the racemate.

Of course, all diastereomers of tolperisone and the acids which themselves bear an asymmetry center are also the subject of the present Application and belong to the scope of the present invention. Such acids are, for example, mandelic acid, hydroxybutyric acids, lactic acid and malic acid.

Another subject of the present invention is also a process for the preparation of addition salts according to the invention of 2,4′-dimethyl-3-piperidinopropiophenone (tolperisone) of Formula (A)

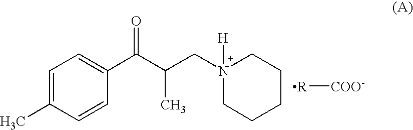

wherein one reacts tolperisone of Formula (B)

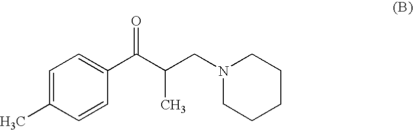

with an organic acid of Formula (C)

R—COOH (C)

in the solvent 2-propanol to form an addition salt of the tolperisone.

Particularly preferred is a process according to the invention, wherein citric acid is used as the organic acid. Therefore, it is preferred that the tolperisone is utilized as a racemate. It is particularly preferred that the (S)-enantiomer of the tolperisone is utilized. However, it is also particularly preferred that the (R)-enantiomer of the tolperisone is utilized.

The process makes it possible to convert both a racemic mixture of tolperisone as well as the pure enantiomers into corresponding addition salts.

The phrase used herein of physiologically compatible organic acid is a phrase familiar to the person skilled in the art. Understood here are all organic acids which are physiologically unobjectionable in the concentrations and quantities used in the form of the addition salts, thus, for example, they do not have toxic or irritating actions nor other effects that in any way adversely affect the health of the patient.

The process according to the invention can be reproduced as follows:

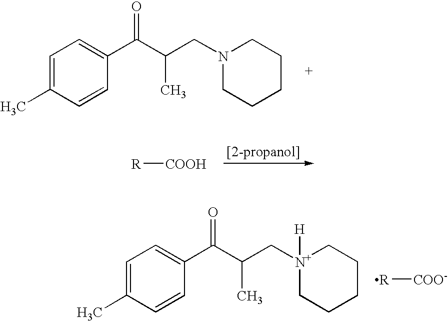

Tolperisone as a racemic mixture or an enantiomer of tolperisone along with an organic acid and the solvent 2-propanol are utilized as the initial substances.

In the process according to the invention, the use of the organic acid, preferably citric acid, is advantageous, because its salts are essentially more stable than tolperisone hydrochloride. Citrate is added as an auxiliary agent to the commercial preparations containing tolperisone hydrochloride in order to obtain a lower pH. Citrate is omitted as an auxiliary agent when tolperisone citrate is used as the active ingredient.

The synthesis of addition salts of tolperisone containing organic acids was not possible with the previously described processes. Surprisingly, the crystallization of tolperisone hydrochloride was successful when the solvent 2-propanol was used.

The method according to the invention makes possible the preparation of addition salts of (R)-tolperisone, of (S)-tolperisone or a racemic mixture of both enantiomers. Therefore, the possibility exists of utilizing the salts of the tolperisone enantiomers both in pure form as well as in any type of mixture in therapeutic preparations. The selectivity of the pharmacological effects can be increased by the use of optically active forms.

Another subject of the present invention is the use of an addition salt according to the invention for the treatment of painful spasms, tensing of the musculature, cervical syndrome, cervicobrachial syndrome, lumbar syndrome, osteoporosis, arthroses of the large joints, rheumatic diseases, fibromyalgia syndrome, chronic polyarthritis, overstressing caused by occupation and sports.

Another subject of the present invention is the use of an addition salt according to the invention for the preparation of a pharmaceutical for the treatment of painful spasms, tensing of the musculature, cervical syndrome, cervicobrachial syndrome, lumbar syndrome, osteoporosis, arthroses of the large joints, rheumatic diseases, fibromyalgia syndrome, chronic polyarthritis, overstressing caused by occupation and sports.

A subject of the present invention is also pharmaceuticals which contain at least one addition salt of tolperisone according to the invention in addition to pharmaceutically acceptable auxiliary agents and/or vehicles.

The subject of the present invention in particular also includes pharmaceuticals for oral, rectal, topical (cutaneous, transdermal, local), subcutaneous, intravenous or intramuscular application, which contain a compound of the general Formula (A) of active ingredient in addition to the usual vehicles and diluting agents.

It is therefore particularly preferred that the tolperisone salts containing organic acid according to the invention can be used without the use of another penetration enhancer. The polybasic acids, such as tartaric acid or citric acid, which are usually added to transdermal systems, act as penetration enhancers. It was now surprisingly established that when the corresponding tolperisone salt with these polybasic acids was used, this enhancer can be omitted. This leads to considerable advantages in the preparation and application of such transdermal systems.

The pharmaceuticals of the invention are prepared in the known way with the usual solid or liquid vehicles or dilution agents and the usually used technical-pharmaceutical auxiliary agents corresponding to the desired type of application with a suitable dosage. The preferred preparations consist of a form of administration which is suitable for oral application. Such forms of administration include, for example, tablets, film tablets, coated tablets, capsules, pills, powders, solutions or suspensions or slow-release forms.

Topical application can be provided, for example, in the form of salves, creams, gels, solutions or by plasters.

Of course, parenteral preparations such as injection solutions are also considered. In addition, suppositories can also be named as preparations, for example.

Corresponding tablets can be obtained, for example, by mixing the active ingredient with known auxiliary agents, for example, inert dilution agents such as dextrose, sugar, sorbitol, mannitol, polyvinylpyrrolidone, bursting agents such as corn starch or alginic acid, binding agents such as starch or gelatins, lubricants such as magnesium stearate or talcum and/or agents for achieving a slow-release effect, such as carboxyl polymethylene, carboxylmethyl cellulose, cellulose acetate phthalate or polyvinyl acetate. The tablets may also consist of several layers.

Correspondingly, coated tablets can be prepared by coating with agents, for example polyvinylpyrrolidone or shellac, gum Arabic, talcum, titanium dioxide or sugar, which are usually used in pill coating by the coating of cores that have been prepared analogously to tablets. In this way, the coated tablet envelope may also consist of several layers, wherein the above-mentioned auxiliary agents given for tablets can be used.

Solutions or suspensions containing the active ingredient used according to the invention may additionally contain a taste-improving agent such as saccharin, cyclamate or sugar, as well as flavorings such as vanilla or orange extract. They may also contain auxiliary agents for inducing suspension, such as sodium carboxymethylcellulose or preservatives such as p-hydroxybenzoate. Capsules containing active ingredients can be prepared, for example, by mixing the active ingredient with an inert vehicle such as lactose or sorbitol and encapsulating in gelatin capsules.

Suitable suppositories can be prepared, for example, by mixing with vehicles provided for this purpose, such as neutral fats or polyethylene glycol or their derivatives.

The preparation of pharmaceuticals according to the invention for topical application is known to the person skilled in the art. Auxiliary agents and enhancers that are known in and of themselves are used in the preparation of the pharmaceuticals for transdermal application according to the invention.

The production of the pharmaceutical preparations according to the invention is known in and of itself and is described in handbooks known to the person skilled in the art, for example Hager's Handbuch [Handbook] (5^{th} ed.) 2, 622-1045; List et al., Arzneiformenlehre [Science of drug form], Stuttgart: Wiss. Verlagsges. [Science Publ. Co.] 1985; Sucker et al., Pharmazeutische Technologie [Pharmaceutical Technology], Stuttgart: Thieme 1991; Ullmann's Enzyklopadie [Encyclopedia] (5^{th} ed.) A 19, 241-271; Voigt, Pharmazeutische Technologie [Pharmaceutical Technology], Berlin: Ullstein Mosby 1995.

The following Examples describe the invention in more detail:

### EXAMPLE 1

### Preparation of Tolperisone Citrate

3.84 g of citric acid (anhydrous), 4.91 g of tolperisone base and 25 ml of 2-propanol were heated under reflux and stirred for 3 minutes in a reaction vessel. The clear solution that was thus obtained was cooled to room temperature and mixed with 25 ml of 2-propanol. The solution was stirred for 15 minutes at room temperature and subsequently incubated for one hour at −15° C. The precipitate was aspirated, washed with 3 ml of 2-propanol and air-dried.

Yield: 7.45 g of white crystals

Melting point: 128.7° C.

¹H-NMR: The results of the ¹H-NMR analysis confirmed the identity of the product and produced a purity of >99%.

### EXAMPLE 2

### Preparation of (S)-Tolperisone Citrate

1.87 g of citric acid (anhydrous), 2.39 g of (S)-tolperisone base and 13 ml of 2-propanol were heated under reflux and stirred for 3 minutes in a reaction vessel. The clear solution that was thus obtained was cooled to room temperature.

Crystallization was induced by rubbing with a glass rod on the vessel wall. Then 8 ml of 2-propanol were added. The solution was incubated overnight at −15° C. The precipitate was aspirated, washed with 3 ml of 2-propanol and air-dried.

Yield: 2.65 g of white crystals

Melting point: 125.2° C.

Optical rotation: 2.0°

¹H-NMR: The spectrum of the ¹H-NMR analysis confirmed the identity of the product.

### EXAMPLE 3

### Preparation of (R)-Tolperisone Citrate

1.92 g of citric acid (anhydrous) and 25 ml of 2-propanol were heated under reflux and stirred for 5 minutes in a reaction vessel. 2.39 g of (R)-tolperisone base were added to the clear solution that formed. The reaction mixture was cooled to room temperature within 10 minutes while stirring. The reaction mixture was stirred on ice for another 30 minutes. The precipitate was aspirated, washed with 3 ml of 2-propanol and air-dried.

Yield: 2.4'5 g of white crystals

Melting point: 124.6° C.

Optical rotation: −2.9°

¹H-NMR: The spectrum of the ¹H-NMR analysis confirmed the identity of the product.

### EXAMPLE 4

### Preparation of Tolperisone Malate

15 ml of ethyl acetate were heated to boiling in a reaction vessel. 0.67 g of L-malic acid was dissolved in the boiling ethyl acetate. Subsequently, 1.23 g of tolperisone base were added. The solution was heated to reflux temperature and 5 ml of ethyl acetate were added. The clear solution thus obtained was stirred for 30 min without additional input of heat and subsequently incubated for 30 min at −15° C. The precipitate was aspirated, washed with 4 ml of ethanol and dried.

Yield: 1.6 g of white crystals

Melting point: 103.9° C.

¹H-NMR: The results of the ¹H-NMR analysis confirmed the identity of the product and produced a purity of >99%.

1. An addition salt of 2,4′-dimethyl-3-piperidinopropiophenone (tolperisone) of Formula (A) 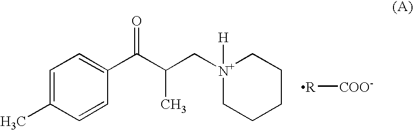
wherein
R is the organic group of a physiologically compatible organic acid.

2. The addition salt according to claim 1, further characterized in that R is an aliphatic, saturated or unsaturated group with up to 5 C atoms, which is optionally substituted with one or more hydroxy, oxo and/or carboxy groups.

3. The addition salt according to claim 1, further characterized in that R is an aryl or aralkyl group, wherein the group contains 5 to 9 C atoms and is optionally substituted with one or more hydroxy and/or carboxy groups.

4. The addition salt according to claim 1, further characterized in that the physiologically compatible organic acid is selected from acetic acid, propionic acid, malonic acid, oxalic acid, gluconic acid, succinic acid, maleic acid, fumaric acid, lactic acid, tartaric acid, malic acid, citric acid, pyruvic acid, hydroxybutyric acids, adipic acid, salicylic acid, phthalic acid, mandelic acid and benzoic acid.

5. The addition salt according to claim 1, further characterized in that the physiologically compatible organic acid is citric acid.

6. The addition salt according to claim 1, namely
tolperisone citrate,
(S)-tolperisone citrate or
(R)-tolperisone citrate.

7. A process for the preparation of addition salts of 2,4′-dimethyl-3-piperidinopropiophenone (tolperisone) of Formula (A) 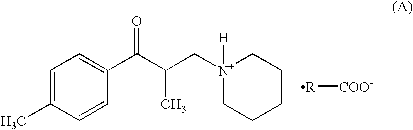
wherein one reacts tolperisone of Formula (B) 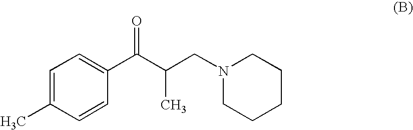
with an organic acid of Formula (C)
R—COOH (C)
in the 2-propanol solvent to form an addition salt of the tolperisone.

8. The process according to claim 7, further characterized in that citric acid is used as the organic acid.

9. The process according to claim 7, further characterized in that tolperisone is utilized as a racemate.

10. The process according to claim 7, further characterized in that the (S)-enantiomer of the tolperisone is utilized.

11. The process according to claim 7, further characterized in that the (R)-enantiomer of the tolperisone is utilized.

12. Use of an addition salt according to claim 1 for the treatment of painful spasms, tensing of the musculature, cervical syndrome, cervicobrachial syndrome, lumbar syndrome, osteoporosis, arthroses of the large joints, rheumatic diseases, fibromyalgia syndrome, chronic polyarthritis, overstressing caused by occupation and sports.

13. The use of an addition salt according to claim 1 for the preparation of a pharmaceutical for the treatment of painful spasms, tensing of the musculature, cervical syndrome, cervicobrachial syndrome, lumbar syndrome, osteoporosis, arthroses of the large joints, rheumatic diseases, fibromyalgia syndrome, chronic polyarthritis, overstressing caused by occupation and sports.

14. Pharmaceuticals, containing an addition salt according to claim 1 in addition to pharmaceutically acceptable auxiliary agents and/or vehicles.

15. The pharmaceuticals according to claim 14, further characterized in that these are present in the form of transdermal systems that do not contain other penetration enhancers.
